(19) United States
(12) Patent Application Publication
(10) Pub. No.: US 2015/0190440 A1
(43) Pub. Date: Jul. 9, 2015

(54) BLOOD PRESSURE REDUCING COMPOSITION FABRICATED BY USING MONASCUS PURPUREUS NTU 568 AND PRIMER FOR THE MONASCUS PURPUREUS NTU 568

(71) Applicant: Sunway Biotech Co., LTD, Taipei (TW)

(72) Inventors: Tzu-Ming Pan, Taipei (TW); Cheng-Lun Wu, Taipei (TW)

(73) Assignee: Sunway Biotech Co., LTD, Taipei (TW)

(21) Appl. No.: 14/255,470

(22) Filed: Apr. 17, 2014

(30) Foreign Application Priority Data
Jan. 6, 2014 (CN) 201410004841.X

(51) Int. Cl. A61K 36/062; C12Q 1/68; A61K 36/8945

(52) U.S. Cl. CPC A61K 36/062; A61K 36/8945; C12Q 1/6895; C12Q 2600/158

(57) ABSTRACT

The present invention relates to a blood pressure reducing composition and primers for Monascus purpureus NTU 568, wherein the composition is a red mold dioscorea (RMD) manufacturing by way of inoculating a Monascus purpureus NTU 568 to a dioscorea substrate and then treating the inoculated dioscorea with culturing and drying processes. This composition is able to reduce blood pressure and prevent the vascular wall from pathological deterioration; therefore, the composition can be applied to clinical treatment and health food. Moreover, at least one nucleotide sequence for M. purpureus NTU 568 and the primers for the nucleotide sequence are also provided in the present invention in order to facilitate the person skilled in Monascus purpureus related art capable of accomplishing the strain (mutant) identification of the M. purpureus NTU 568.

### SEQUENCE LISTING

The instant application contains a Sequence Listing which has been submitted in ASCII format via EFS-Web and is hereby incorporated by reference in its entirety. The ASCII copy is named sequence.txt and is 5,705 bytes in size.

### BACKGROUND OF THE INVENTION

1. Field of the Invention

The present invention relates to the technology field of blood pressure reducing compositions, and more particularly to a blood pressure reducing composition fabricated by using Monascus purpureus NTU 568 and primers for the Monascus purpureus NTU 568.

2. Description of the Prior Art

Blood pressure (BP), sometimes referred to as arterial blood pressure, is the pressure exerted by circulating blood upon the walls of blood vessels, and is one of the principal vital signs. So that, if the amount of the blood outputted by humane heart increases or the resistance on the blood circulation is enhanced, the blood pressure would then rise correspondingly. Wherein, the enhancement of blood circulation resistance is resulted from the formation of plaque on the vascular wall, such as fat and cholesterol. In addition, over-nervous spirits also induces the rise of the BP. When a man is at an over-nervous state, his cerebral cortex and sympathetic nerve would be respectively excited and activated; and then, the activated sympathetic nerve causes the heartbeat to speed up and the cardiac contractility to increase, such that the amount of the blood outputted by the human heart is increased and then the blood pressure rises.

In recent years, hypertension becomes a conventional chronic disease, wherein the judgment standard for hypertension is to determine whether the systolic blood pressure (SBP) is greater than 140 mmHg or the diastolic blood pressure (DBP) is greater than 90 mmHg, and the hypertension illness sign includes dizziness, headache, palpitation, and hard to breath (dyspnea). Moreover, if one man suffers from the hypertension for a long time, the man may further suffer from other companion diseases, such as stroke, coronary artery heart disease (CAHD), and kidney failure (renal insufficiency). Currently, the hypertension-curing drugs can be divided into diuretic agent (diuretics), sympathetic nerve blocker, angiotensin receptor blocker (ARB), and angiotensin converting enzyme inhibitor (ACEI). However, all the aforesaid hypertension-curing drugs include drawbacks as follows:

(1) To lowering blood pressure, the diuretics, for example thiazide, controls hypertension in part by inhibiting reabsorption of sodium (Na⁺) and chloride (Cl⁻) ions from the distal convoluted tubules in the kidneys by blocking the thiazide-sensitive Na⁺—Cl⁻ symporter. However, human body responds to hypovolemia by opposing diuresis, one effect of which is to produce aldosterone which stimulates the Na/K exchanger, resulting in further loss of potassium called as “hypokalemicnephropathy”.

(2) Sympathetic nerve blocker provides a blockade of beta-receptors in the brainstem and of prejunctional beta-receptors in the periphery inhibits the release of neurotransmitters and decreases sympathetic nervous system activity, so as to reduce the heart rate and the blood pressure. However, the sympathetic nerve blocker induces the side effects such as bradycardia, posture hypotension and asynodia.

(3) Both the ARB and the ACEI are used for avoiding the Renin-angiotensin system (RAS) from overactivity in order to carry out the reducing of the blood pressure. However, both the ARB and the ACEI include a primary side effect of renal failure.

Accordingly, in view of the conventional hypertension-curing drugs still including drawbacks and shortcomings, the inventor of the present application has made great efforts to make inventive research thereon and eventually provided a blood pressure reducing composition fabricated by using Monascus purpureus NTU 568 and primers for the Monascus purpureus NTU 568.

### SUMMARY OF THE INVENTION

The primary objective of the present invention is to provide a blood pressure reducing composition, which is a red mold dioscorea (RMD) manufacturing by way of inoculating a Monascus purpureus NTU 568 to a dioscorea substrate and then treating the inoculated dioscorea substrate with culturing and drying processes, so as to obtained a powdered RMD with a great blood pressure-reducing function.

Accordingly, to achieve the primary objective of the present invention, the inventor of the present invention provides a blood pressure reducing composition, which is a red mold dioscorea (RMD) manufacturing through inoculating a Monascus purpureus NTU 568 to a dioscorea substrate and then treating the inoculated dioscorea substrate with culturing and drying processes; wherein, an specific intake dosage of the blood pressure reducing composition for an adult user used to reduce the systolic blood pressure (SBP) and diastolic blood pressure (DBP) in a short period of 8 hr is ranged from 2.2 g to 11 g.

Moreover, in order to achieve the primary objective of the present invention, the inventor of the present invention further provides a primer for identifying the said Monascus purpureus NTU 568, wherein the being primer is selected from the group consisting of:

(1) primer PKSα F:
(SEQ ID NO 4)
GACTGCGGTCATCCGGCCC;
(2) primer PKSα R:
(SEQ ID NO 5)
GCGTGTCCCCGGAGCTACA;
(3) primer PKSδ F:
(SEQ ID NO 6)
GCGAGCCAACCGTCTGGACC;
(4) primer PKSδ R:
(SEQ ID NO 7)
GCGTGTCCCCGGAGCTACA;
(5) primer PKSγ F:
(SEQ ID NO 8)
GCGAGCCAACCGTCTGGACC;
and
(6) primer PKSγ R:
(SEQ ID NO 9)
CGAGACGACCACCGTTGCCC.

### BRIEF DESCRIPTION OF THE DRAWINGS

The invention as well as a preferred mode of use and advantages thereof will be best understood by referring to the following detailed description of an illustrative embodiment in conjunction with the accompanying drawings, wherein:

FIG. 1 shows a flow chart of a blood pressure reducing composition manufacturing method;

FIG. 2 shows a conserved domain analysis diagram for PKSα nucleotide sequence;

FIG. 3 shows a conserved domain analysis diagram for PKSδ nucleotide sequence;

FIG. 4 shows a conserved domain analysis diagram for PKSγ nucleotide sequence;

FIG. 5A shows statistical data plots for diastolic blood pressure (DBP) of the SHRs and WKYs in the experiment groups listed in Table 6;

FIG. 5B shows statistical data plots for systolic blood pressure (SBP) of the SHRs and WKYs in the experiment groups listed in Table 6;

FIG. 6A shows statistical data plots for diastolic blood pressure (DBP) of the SHRs and WKYs in the experiment groups listed in Table 6;

FIG. 6B shows statistical data plots for systolic blood pressure (SBP) of the SHRs and WKYs in the experiment groups listed in Table 6;

FIG. 7A shows statistical data plots for diastolic blood pressure (DBP) of the SHRs in the C group, M group, 1RM group, and 5RM group been treat with the one single oral administration;

FIG. 7B shows statistical data plots for systolic blood pressure (SBP) of the SHRs in the C group, M group, 1RM group, and 5RM group been treat with the one single oral administration;

FIG. 8A shows statistical data plots for diastolic blood pressure (DBP) of the SHRs in the C group, M group, 1RM group, and 5RM group been treat with the chronic administration experiment;

FIG. 8B shows statistical data plots for systolic blood pressure (SBP) of the SHRs in the C group, M group, 1RM group, and 5RM group been treat with the chronic administration experiment; and

FIG. 9 shows histologic section images of the artery of the SHRs and the WKYs in the experiment group of Table 6.

### DETAILED DESCRIPTION OF THE PREFERRED EMBODIMENTS

To more clearly describe a blood pressure reducing composition fabricated by using Monascus purpureus NTU 568 and primers for the Monascus purpureus NTU 568 according to the present invention, embodiments of the present invention will be described in detail with reference to the attached drawings hereinafter.

Monascus purpureus NTU 568 is an excellent local Monascus purpureus strain, and which is studied and developed by Tzu-Ming PAN, the graduate chair of Institute of Microbiology and Biochemistry of National Taiwan University, and the R&D team thereof. In the present invention, the Monascus purpureus NTU 568 has a specific nucleotide sequence of SEQ ID NO 1, SEQ ID NO 2 or SEQ ID NO 3 is deposited with Deutsche Sammlung von Mikroorganismen and Zellkulturen GmbH (DSMZ, Inhoffenstr. 7B, D-38124 Braunschweig, Germany) on Nov. 18, 2013, with the accession number of DSM 28072. The Monascus purpureus NTU 568 includes the characteristics of: growing rapidly, strong starch hydrolysis, high metabolites production. The basic culture medium for Monascus purpureus NTU 568 needs includes 2% rice powder, and the best culture temperature is 30° C., the best culture time is 48 hours and the best culture pressure is 1 atm.

To verify the viability of Monascus purpureus NTU 568, the strain of Monascus purpureus NTU 568 is moved from a slant tube to a culture medium of potato dextrose agar (PDA) for culturing process. After 15-day culture, it digs and takes out three mycelium with the size of 1 cm³ from the PDA, and then disposes the three mycelium into a culture fluid having 2% rice powder for next-stage culture. Therefore, after 48-hour culture, the Monascus purpureus NTU 568 reveals high viability because the culture fluid shows red color. Herein, it needs to further explain that, the storage method for Monascus purpureus NTU 568 is to culture the Monascus purpureus NTU 568 on a PDA medium disposed in a slant tube under the store temperature of 4° C.; moreover, the Monascus purpureus NTU 568 must be treated with one time sub-cultured per 3 months.

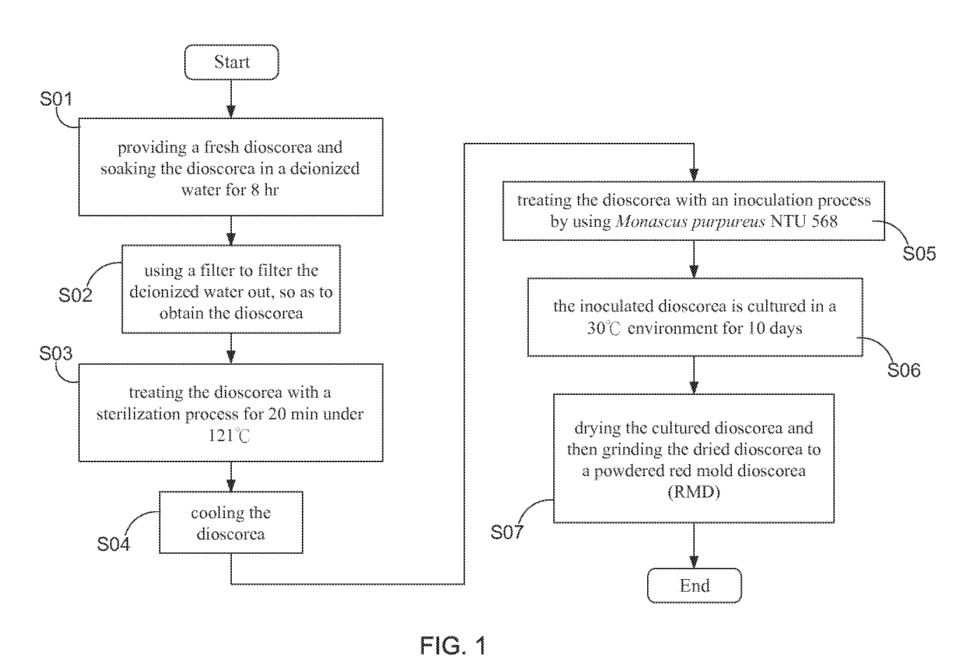

Continuously, the Monascus purpureus NTU 568 is used for fabricating a blood pressure reducing composition proposed by the present invention. Please refer to FIG. 1, which illustrate a flow chart of a blood pressure reducing composition manufacturing method, wherein the manufacturing method mainly consists of 7 steps.

First of all, the manufacturing method executes step (S01) for providing a fresh dioscorea and soaking the dioscorea in a deionized water for 8 hr. Next, the manufacturing method executes step (S02) for using a filter to filter the deionized water out, so as to obtain the dioscorea. Subsequently, step (S03) is executed for treating the dioscorea with a sterilization process for 20 min under 121° C. Therefore, the sterilized dioscorea is cooled in step (S04).

After finishing the sterilization process, the manufacturing method continuously executes step (505) for treating the dioscorea with an inoculation process by using Monascus purpureus NTU 568. Therefore, the inoculated dioscorea is cultured in a 30° C. environment for 10 days in step (S06). Eventually, the manufacturing method executes step (S09) for drying the cultured dioscorea and then grinding the dried dioscorea to a powdered red mold dioscorea (RMD).

Particularly, the aforesaid Monascus purpureus NTU 568 used in the step (S03) has a specific nucleotide sequence of SEQ ID NO 1, SEQ ID NO 2 or SEQ ID NO 3 is deposited with Deutsche Sammlung von Mikroorganismen and Zellkulturen GmbH (DSMZ, Inhoffenstr. 7B, D-38124 Braunschweig, Germany) on Nov. 18, 2013, with the accession number of DSM 28072.

Furthermore, in order to identify the DNA sequence of the Monascus purpureus NTU 568, it obtains the whole genome sequence of the Monascus purpureus NTU 568 by way of pyrosequencing, wherein the whole genome sequence of the Monascus purpureus NTU 568 includes 3,326 contigs with the total sequence length of 247,174,841 bps. Moreover, in the 3,326 contigs, the largest length of a specific contig is 175,588 bps.

Next, the Aspergillus is taken as a reference species and the software of FGENESH (SoftBerry, Inc., NY, USA) is then used for analyzing and predicting the DNA sequence of the Monascus purpureus NTU 568. The analysis and predict result shows 8,191 sequence data of mRNA and protein, wherein the total sequence length of the mRNA is 13,140,800 bps. Therefore, the whole genome sequence of the Monascus purpureus NTU 568 and the mRNA and protein sequence data are further edited to a single FASTA file, and then the FASTA file is transformed into a BLAST data by using the software of BLAST⁺ (Boratyn et al., 2013) for executing the gene search and alignment.

The gene search and alignment are executed by using polyketide synthases (PKSs) mechanism and model. Please refer to following table 1, which records several PKS fragments in PKS conserved domain. Therefore, the gene alignment between the PKS fragments of M. pilosus mok A and the BLASTp data of the Monascus purpureus NTU 568 as well as the BLASTn data of the Monascus purpureus NTU 568 have been completed.

TABLE 1

| Accession | | |
|---|---|---|
| no. | Description | Sequence |
| cd00833 | a polyketide synthases | IAIVGMACRFPGAADPDE |
| | (PKSs) polymerize simple fatty | FWENLLEGRDAISEIPEDRWDA |
| | acids into a large variety of | DGYYPDPGKPGKTYTRRGGFL |
| | different products, called | DDVDAFDAAFFGISPREAEAM |
| | polyketides, by successive | DPQQRLLLEVAWEALEDAGYS |
| | decarboxylating Claisen | PESLAGSRTGVFVGASSSDYLE |
| | condensations. | LLARDPDEIDAYAATGTSRAFL |
| | PKSs can be divided into 2 | ANRISYFFDLRGPSLTVDTACSS |
| | groups, modular type I PKSs | SLVALHLACQSLRSGECDLALV |
| | consisting of one or more large | GGVNLILSPDMFVGFSKAGML |
| | multifunctional proteins and | SPDGRCRPFDADADGYVRGEG |
| | iterative type II PKSs, complexes | VGVVVLKRLSDALRDGDRIYA |
| | of several monofunctional | VIRGSAVNQDGRTKGITAPSGE |
| | subunits. | AQAALIRRAYARAGVDPSDID |
| | | YVEAHGTGTPLGDPIEVEALA |
| | | KVFGGSRSADQPLLIGSVKSNI |
| | | GHLEAAAGLAGLIKVVLALEH |
| | | GVIPPNLHFETPNPKIDFEESPL |
| | | RVPTEARPWPAPAGPRRAGVSS |
| | | FGFGGTNAHVIL |
| DQ176595 | PKS domain sequence of | ACGACATCGTAGGGGGT |
| | polyktide synthase mokA of | GCGTTCGCGAGTCGCGATGAC |
| | monacolin K biosynthetic gene | CTCGGTCATCTTGGCGCTGCC |
| | cluster in M.pilosus | AATCGAACCACTCTCCGCCTG |
| | | GCCCTGCTTGTAATCGAAGAC |
| | | CGCTTGGAACAAGGGGGCCG |
| | | GTTCCGCTGTTTCGGCGGTGG |
| | | CCCCCGGGACCTCGAATCCGA |
| | | GGCGCTCGAGCAGCACCCCG |
| | | TAGGGCACGCGGGCGTGCTG |
| | | CATGGCCTCGCGCACCTTGTC |
| | | CTTGGTGGCGACCAGGTGCTC |
| | | GCCAAAGGTGATGTGCGGGA |
| | | CGAAGTTGCGGAAGCGCAGC |
| | | GGGAGCAGGTTGGCGAAAAA |
| | | GCCCATGCCCGCCAGTTCATC |
| | | CACGTTCGTGCGATTGGTGTC |
| | | GGCCAGGCCTATGCTGAAGTC |
| | | GCTGCTGCCCGTCAATCGTGC |
| | | CAGGAGCACGTGGTACGCAG |
| | | CCAGGTAGAATTGCATGGGCG |
| | | TGGCTTTGTGCTTGCGACTGC |
| | | GCTCGCGGATGCGGAAAGCG |
| | | ACCATGGGGTCGAGACGCGC |
| | | GATCGCTTCGTGTTGCTTCCA |
| | | CGAGTTGGGCTGGCGGGCGT |
| | | GGTTCGGGCTATTAAGGCCAT |
| | | CTTCGCCCAGAAGCATCCGCG |
| | | GGAGGACCGGGGACACCACG |
| | | CCCGTGGGCTGGTGGTGCATC |
| | | GATTCCCAGTACGCGAGGTCC |
| | | GCATCCATCTGGCCGGACTCG |
| | | AGCGCTTCTCGCTGCCGCGTC |
| | | GCGAGGTCTGCAAATTGAGG |
| | | GACGTGCTTGTCGAGGGTCA |
| | | CGCCGCCGTATAACTGGCTCG |
| | | CTTCGACAAAGATATT |

Therefore, the gene alignment results reveal that, besides the well-known PKS genomes of citrinin (Accession: AB243687.1), monacolin K (Accession: DQ176595.1) and PKS1 (Accession: AJ414729.1), the whole genome sequence of the Monascus purpureus NTU 568 further includes 7 candidate gene fragment in PKS conserved domain, wherein the 7 candidate gene fragment are named as PKSε, PKSθ, PKSγ, PKSκ, PKSδ, PKSα, and PKSσ recorded in following table 2. Moreover, after completing the DELTA-BLAST analysis, the PKS fragments of PKSγ, PKSδ and PKSα are regarded as new PKS fragments of M. purpureus which are never recorded or written in any literatures or data base.

TABLE 2

| PKS | Contig | | |
|---|---|---|---|
| ID | no. | Protein sequence ID | E value |
| PKSε | 986 | 148__exon_(s)_431197__−_ | 1e-102 |
| | | 443034__3945_aa,_chain_+ | |
| PKSθ | 195 | 1001__12_exon_(s)_2896331__−_ | 1e-102 |
| | | 2908604_ _3854_aa,_chain_+ | |
| PKSΥ | 549 | 535__6_exon_(s)_1607184__−_ | 7e-98 |
| | | 1614486_ _2307_aa,_chain_− | |
| PKSκ | 1154 | 396__5_exon_(s)_1203158__−_ | 6e-92 |
| | | 1210134__2245_aa,_chain_+ | |
| PKSδ | 977 | 403__6_exon_(s)_1222398__−_ | 7e-77 |
| | | 1229259__2188_aa,_chain_+ | |
| PKSα | 657 | 38__6_exon_(s)_101837__−_ | 9e-72 |
| | | 106246__1263_aa,_chain__− | |
| PKSσ | 200 | 757__13_exon_(s)_2356480__−_ | 5e-59 |
| | | 2361939__1583_aa,_chain_− | |

Based above gene search and alignment results, it is able to assume that the gene fragment of PKSα may be a novel gene fragment (sequence) for the Monascus purpureus NTU 568. Therefore, as listed in the following Sequence Listing, the nucleotide sequence of PKSα is defined as SEQ ID NO 1, and the sequence length of the nucleotide sequence of SEQ ID NO 1 is 1,390 bps. Furthermore, the nucleotide sequence of PKSα is treated with a BLASTx sequence alignment, and the alignment results are recorded in following table 3. Moreover, please refer to

TABLE 3

| | | Max | |
|---|---|---|---|
| | | identity | |
| Accession no. | Description | (%) | E value |
| XP_002149769 | PKS: Talaromyces | 64.2 | 0 |
| | marneffei ATCC 18224 | | |
| XP_002340038 | PKS: Talaromyces | 63.2 | 0 |
| | stipitatus ATCC 10500 | | |
| EFW23245 | PKS: Coccidioides | 60.5 | 0 |
| | posadasii str. Silveira | | |
| XP_003070229 | PKS: Coccidioides | 60.4 | 0 |
| | posadasii C735 delta | | |
| | SOWgp | | |
| EJB11047 | citrinin (PKS): | 60.3 | 0 |
| | Coccidioides immitis | | |
| | C735 RS | | |
| XP_001243185 | hypothetical protein | 60.3 | 0 |
| | (CIMG_07081): | | |
| | Coccidioides immitis RS | | |
| XP_002487778 | PKS: Talaromyces | 59.3 | 0 |
| | stipitatus ATCC 10500 | | |
| EOD53036 | putative polyketide | 57.8 | 0 |
| | synthase protein: | | |
| | Neofusicoccum parvum | | |
| | UCRNP2 | | |
| CAK40124 | unnamed protein | 58.8 | 0 |
| | product: Aspergillus | | |
| | niger | | |
| XP_001393501 | polyketide | 58.8 | 0 |
| | synthase: Aspergillus | | |
| | niger CBS 513.88 | | |

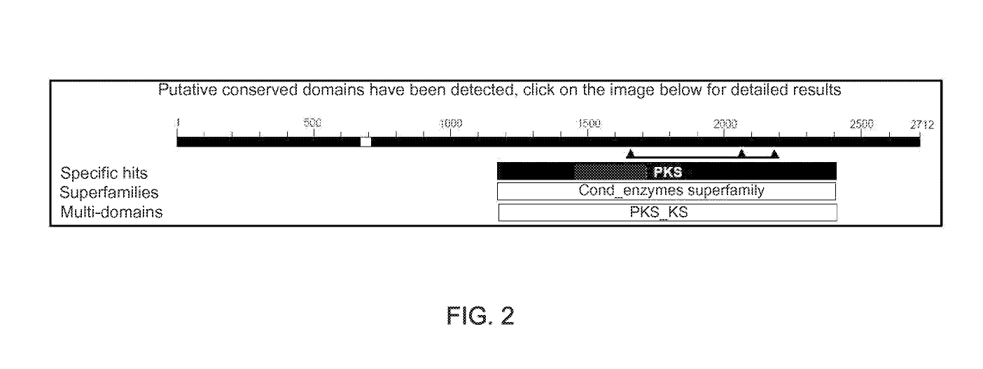

Continuously, please refer to FIG. 2, there is shown a conserved domain analysis diagram for PKSα nucleotide sequence. From FIG. 2, it is able to know that the conserved domain PKS of PKSα is PKS_KS, which belongs to type II polyketide synthases (PKS). Moreover, from the table 3, it can further find that the PKS most similar to the PKSα is Talaromyces marneffei ATCC 1822 (identity=64.2), and there has no PKSs of Monascus genus similar or the same to the PKSα. So that, it is able to confirm that the gene fragment of PKSα is a novel gene fragment (sequence) for the Monascus purpureus NTU 568 based above comparison and analysis.

Moreover, the gene fragment of PKSδ can also be assumed as a novel gene fragment (sequence) for the Monascus purpureus NTU 568. As listed in the following Sequence Listing, the nucleotide sequence of PKSδ is defined as SEQ ID NO 2, and the sequence length of the nucleotide sequence of SEQ ID NO 2 is 1,024 bps. In order to identify whether the assumption is correct or not, the nucleotide sequence of PKSδ is treated with a BLASTx sequence alignment, and the alignment results are recorded in following table 4.

TABLE 4

| | | Max | |
|---|---|---|---|
| | | identity | |
| Accession no. | Description | (%) | E value |
| XP_001270321 | PKS: Aspergillus clavatus | 80.1 | 0 |
| | NRRL 1 | | |
| ENH62327 | Lovastatin nonaketide | 39.1 | 0 |
| | synthase: Fusarium | | |
| | oxysporum f. sp. cubense | | |
| | race 1 | | |
| EKV12048 | Phenolpthiocerol synthesis | 36.9 | 0 |
| | polyketide synthase ppsA: | | |
| | Penicillium digitatum | | |
| | PHI26 | | |
| ELA32194 | polyketide synthase: | 36.5 | 0 |
| | Colletotrichum | | |
| | gloeosporioides Nara gc5 | | |
| ELA38363 | polyketide synthase: | 37.3 | 0 |
| | Colletotrichum | | |
| | gloeosporioides Nara gc5 | | |
| EKV06858 | hypothetical protein | 34.7 | 0 |
| | PDIG_76310: Penicillium | | |
| | digitatum PHI26 | | |
| EFQ35173 | containing protein: | 36.6 | 0 |
| | Glomerella graminicola | | |
| | M1.001 | | |
| XP_664395 | hypothetical protein | 34.3 | 0 |
| | AN6791.2: Aspergillus | | |
| | nidulans FGSC A4 | | |
| ENH88027 | polyketide synthase: | 37.1 | 0 |
| | Colletotrichum orbiculare | | |
| | MAFF 240422 | | |
| ELQ32864 | fatty acid synthase | 37.8 | 0 |
| | S-acetyltransferase: | | |
| | Magnaporthe oryzae Y34 | | |

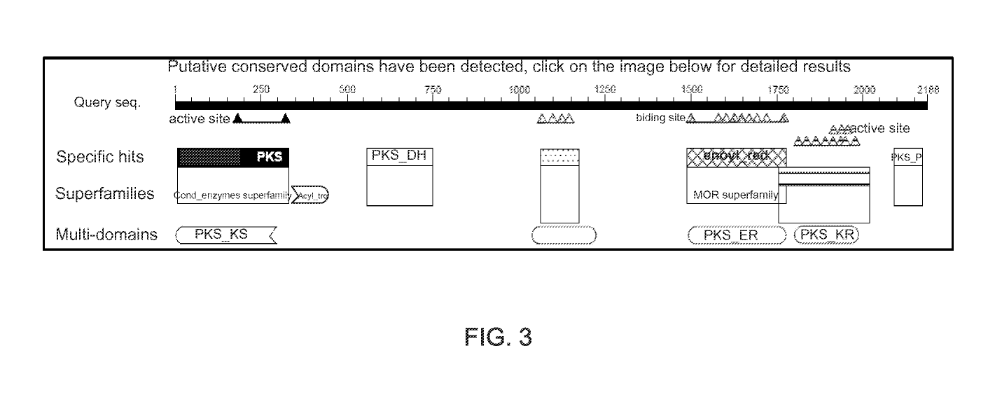

Please refer to FIG. 3, there is shown a conserved domain analysis diagram for PKSδ nucleotide sequence. From FIG. 3, it is able to know that the conserved domain PKS of PKSδ is PKS_KS-DH-MT-ER-KR-ACP, which belongs to type I polyketide synthases (PKS). Moreover, from the table 4, it can further find that the PKS most similar to the PKSδ is the polyketide synthases (PKS) of Aspergillus clavatus NRRL 1 (identity=80.1), and there has no PKSs of Monascus genus similar or the same to the PKSδ. So that, it is able to confirm that the gene fragment of PKSδ is a novel gene fragment (sequence) for the Monascus purpureus NTU 568 based above comparison and analysis.

Besides, the gene fragment of PKSγ can also be assumed as a novel gene fragment (sequence) for the Monascus purpureus NTU 568. As listed in the following Sequence Listing, the nucleotide sequence of PKSγ is defined as SEQ ID NO 3, and the sequence length of the nucleotide sequence of SEQ ID NO 3 is 1,096 bps. In order to identify whether the assumption is correct or not, the nucleotide sequence of PKSγ is treated with a BLASTx sequence alignment, and the alignment results are recorded in following table 5.

TABLE 5

| | | Max | |
|---|---|---|---|
| | | identity | |
| Accession no. | Description | (%) | E value |
| XP_002485355 | PKS: Talaromyces | 44.8 | 0 |
| | stipitatus ATCC 10500 | | |
| ADA79525 | PKS: Delitschia winteri | 44.9 | 0 |
| XP_001273762 | PKS: Aspergillus clavatus | 45.1 | 0 |
| | NRRL 1 | | |
| XP_002482833 | PKS: Talaromyces | 44.4 | 0 |
| | stipitatus ATCC 10500 | | |
| XP_001258783 | PKS: Neosartorya fischeri | 45.5 | 0 |
| | NRRL 181 | | |
| XP_001816573 | PKS: Aspergillus oryzae | 44.8 | 0 |
| | RIB40 | | |
| EDP53518 | PKS: Aspergillus fumigatus | 45.8 | 0 |
| | A1163 | | |
| XP_748462 | PKS: Aspergillus fumigatus | 45.6 | 0 |
| | Af293 | | |
| BAE54571 | unnamed protein product: | 44.2 | 0 |
| | Aspergillus oryzae | | |
| | RIB40 | | |
| XP_002383534 | PKS: Aspergillus flavus | 44.3 | 0 |
| | NRRL3357 | | |

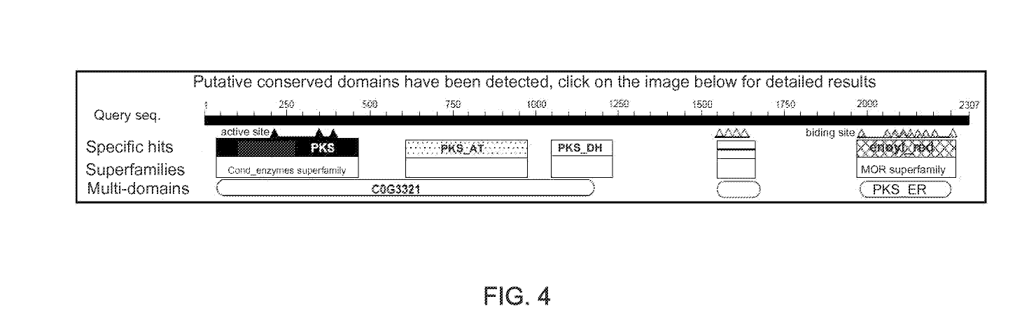

Please refer to FIG. 4, there is shown a conserved domain analysis diagram for PKSγ nucleotide sequence. From FIG. 4, it is able to know that the conserved domain PKS of PKSγ is PKS_KS-DH-MT-ER, which belongs to type I polyketide synthases (PKS). Moreover, from the table 5, it can further find that the PKS most similar to the PKSγ is the polyketide synthases (PKS) of Talaromyces stipitatus ATCC 10500 (identity=44.8), and there has no PKSs of Monascus genus similar or the same to the PKSγ. So that, it is able to confirm that the gene fragment of PKSγ is a novel gene fragment (sequence) for the Monascus purpureus NTU 568 based above comparison and analysis.

Thus, through above descriptions, the novel gene fragments and the related nucleotide sequence of the Monascus purpureus NTU 568 have been introduced. Moreover, the powdered red mold dioscorea (RMD) manufacturing by way of inoculating a Monascus purpureus NTU 568 to a dioscorea substrate and then treating the inoculated dioscorea substrate with culturing and drying processes possesses the functionality to reduce the blood pressure. In which, if a daily diet amount of an adult user includes the powdered RMD with a specific weight percent ranged between 0.2 wt % and 0.25 wt %, then the systolic blood pressure (SBP) and diastolic blood pressure (DBP) of the adult user can be chronically lowered. Besides, if the adult user intakes the powdered RMD with an specific intake dosage ranged between 2.2 g and 11 g, then the systolic blood pressure (SBP) and diastolic blood pressure (DBP) of the adult user can be chronically reduced in a short period of 8 hr.

In order to prove the blood pressure reducing efficiency of the blood pressure reducing composition (i.e., the RMD) proposed by the present invention, several experiments have completed and a variety of experiment data will be presented in following paragraphs. Please refer to following Table 6, which integrate with a plurality of experiment groups.

TABLE 6

| | | Main | Extract |
|---|---|---|---|
| | | feeding | feeding stuffs |
| Group | Rats | stuffs | for experient |
| WC | WKY | Chew diet | water |
| W1R | WKY | + | 1-fold (1X) red mold |
| | | water | dioscorea (RMD) |
| | | | (176 mgkg-1day-1) |
| W5R | WKY | | 5-fold (5X) RMD |
| | | | ((176 × 5) mgkg-1day-1) |
| C | SHR | | water |
| 1R | SHR | | 1-fold (1X) RMD |
| 5R | SHR | | 1-fold (5X) RMD |
| M | SHR | | amlodipine |
| | | | (0.4 mgkg-1day-1) |
| 1RM | SHR | | amlodipine + 1-fold |
| | | | (1X) RMD |
| 5RM | SHR | | amlodipine + 1-fold |
| | | | (5X) RMD |

Spontaneous hypertensive rats (SHRs) and Wistar-Kyoto strains of normotensive rats (WKYs) are chosen to be experiment animals, wherein the SHRs would spontaneously suffer from the hypertension when they grow to 5˜6 weeks old. Herein, 8-week old SHRs are used for carrying out the experiments, and these 8-week old SHRs and WKYs are pre-fed with chew diet and water for 5 weeks before starting the experiments. Herein, it needs to further explain that, the experiment groups listed in Table 6 are divided to 9 groups. The rats in the 9 groups are fed with the main feeding stuffs (i.e., chew diet and water) during first week to 13-th week. Moreover, the rats in the 9 groups are fed with the main feeding stuffs as well as the extract feeding stuffs at 14-th week in order to accomplish one single oral administration experiments; therefore, the rats in the 9 groups are continuously fed with the main feeding stuffs as well as the extract feeding stuffs starting from 14-th week and continuing for 8 weeks, so as to carry out one chronic administration experiment. The designed experiment groups consist of:

(1) WKYs control (WC) group: the WKYs in WC group are pre-fed for 5 weeks, and the WKYs are fed with the extract feeding stuffs of water via gastric tube starting from 14-th week;

(2) W1R group: the WKYs in W1R group are pre-fed for 5 weeks, and the WKYs are fed with the extract feeding stuffs of 1×RMD via gastric tube starting from 14-th week;
(3) W5R group: the WKYs in W5R group are pre-fed for 5 weeks, and the WKYs are fed with the extract feeding stuffs of 5×RMD via gastric tube starting from 14-th week;

(4) Control (C) group: the SHRs in C group are pre-fed for 5 weeks, and the SHRs are fed with the extract feeding stuffs of water via gastric tube starting from 14-th week;

(5) 1R group: the SHRs in 1R group are pre-fed for 5 weeks, and the SHRs are fed with the extract feeding stuffs of 1×RMD via gastric tube starting from 14-th week;
(6) 5R group: the SHRs in 5R group are pre-fed for 5 weeks, and the SHRs are fed with the extract feeding stuffs of 5×RMD via gastric tube starting from 14-th week;

(7) M group: the SHRs in M group are pre-fed for 5 weeks, and the SHRs are fed with the extract feeding stuffs of amlodipine via gastric tube starting from 14-th week;

(8) 1RM group: the SHRs in 1RM group are pre-fed for 5 weeks, and the SHRs are fed with the extract feeding stuffs of amlodipine and 1×RMD via gastric tube starting from 14-th week;

(9) 5RM group: the SHRs in 5RM group are pre-fed for 5 weeks, and the SHRs are fed with the extract feeding stuffs of amlodipine and 5×RMD via gastric tube starting from 14-th week;

According to the body surface area (BSA) equation provided by Food and Drug Administration (FDA), the BSA of a standard man with the body height of 170 cm and the body weight of 65 kg can be calculated through the mathematical calculation formula of BSA (m²)=0.003207{H^{0.3}×W^{[0.07285-(0.0188×LOG (w))}]}, wherein the BSA value obtained from aforesaid calculation formula for the standard man is 1.762 m². On the other hand, the BSA value for the SHRs can also be calculated through the mathematical calculation formula of BSA (m²) (8.99 W^{0.6899})/100, and the BSA value is 0.049 m². Moreover, because the experiments use “2.2 g” as a standard feeding dosage, the “1-fold” in Table 6 can be calculated to 176 mgkg⁻¹day⁻¹ according to BSA value of the SHRs. Herein, it needs to further explain that, M group is taken as a positive control group because amlodipine is a well-known blood pressure lowering substance. In the experiments, the lowest effective dosage (i.e., 1-fold dosage) for amlodipine is defined to 0.4 mgkg⁻¹day⁻¹.

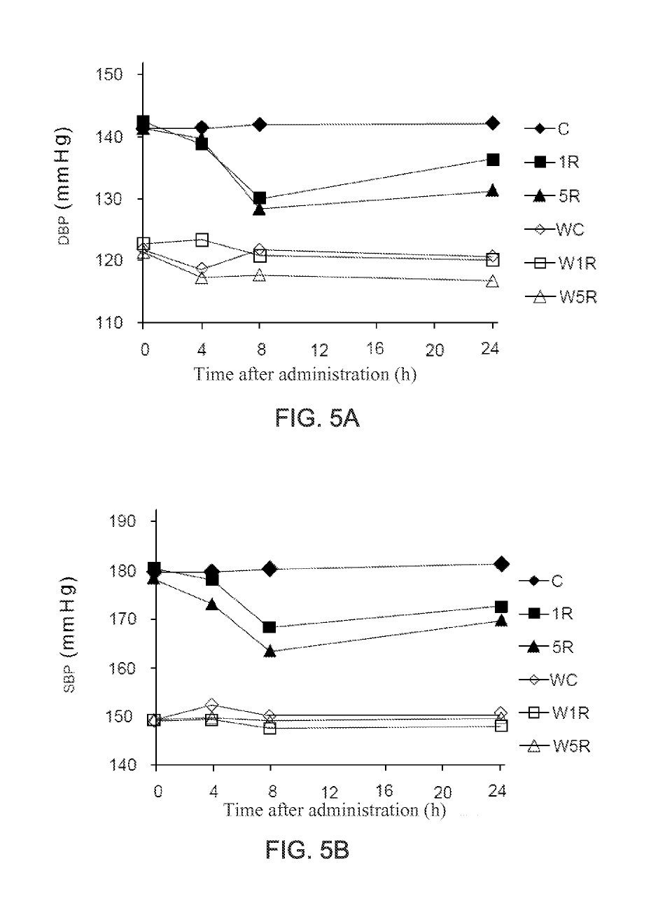

Please refer to FIG. 5A and FIG. 5B, there are respectively shown statistical data plots for systolic blood pressure (SBP) and diastolic blood pressure (DBP) of the SHRs and WKYs in the experiment groups listed in Table 6 and been treat with the one single oral administration. The SBP value and the DBP value of the SHRs in 1R group and 5R group, as shown in FIGS. 5A and 5B, obviously decrease at 8 hr and 24 hr comparing to the SBP value and the DBP value of the SHRs in C group. Moreover, from FIG. 5A and FIG. 5B, it can also find that the DBP value of the WKYs in W1R group and W5R group show no obvious discrepancy comparing to the SBP value and the DBP value of the WKYs in WC group. Therefore, the experiment data of FIG. 5A and FIG. 5B have proven that: (1) the single one oral administration of RMD can indeed lower the BP (blood pressure) value of a spontaneous hypertensive rat; and (2) the single one oral administration of RMD would not affect the BP (blood pressure) value of a normotensive rat. The BP data for SHRs and WKYs in the experiment groups are integrated in following Table 7.

TABLE 7

| | SBP (mmHg) | | | | DBP (mmHg) | | | |
|---|---|---|---|---|---|---|---|---|
| Group | 0 hr | 4 hr | 8 hr | 24 hr | 0 hr | 4 hr | 8 hr | 24 hr |
| WC | 149 ± 4.0 | 153 ± 1.7 | 150 ± 3.3 | 151 ± 2.5 | 122 ± 3.6 | 119 ± 2.6 | 122 ± 3.7 | 121 ± 3.6 |
| W1R | 149 ± 2.7 | 149 ± 3.1 | 148 ± 3.7 | 148 ± 2.6 | 123 ± 2.8 | 123 ± 1.4 | 121 ± 3.6 | 120 ± 2.7 |
| W5R | 149 ± 2.9 | 150 ± 2.4 | 149 ± 4.8 | 150 ± 3.1 | 121 ± 2.9 | 117 ± 3.3 | 118 ± 2.2 | 117 ± 2.6 |
| C | 180 ± 7.5 | 180 ± 4.6 | 180 ± 8.2 | 181 ± 3.2 | 141 ± 4.0 | 141 ± 3.1 | 142 ± 3.6 | 142 ± 4.5 |
| 1R | 180 ± 4.3 | 178 ± 3.6 | 168 ± 2.6* | 173 ± 4.7* | 142 ± 3.3 | 139 ± 4.5 | 130 ± 3.3* | 136 ± 3.5* |
| 5R | 178 ± 4.0 | 173 ± 2.0 | 163 ± 3.2* | 170 ± 2.4* | 141 ± 2.2 | 140 ± 5.3 | 128 ± 3.5* | 131 ± 3.5* |
| M | 180 ± 3.6 | 178 ± 5.4 | 171 ± 3.1 | 175 ± 7.6 | 143 ± 2.9 | 143 ± 3.8 | 134 ± 4.9 | 137 ± 4.2 |
| 1RM | 178 ± 1.9 | 179 ± 4.3 | 164 ± 3.8** | 164 ± 3.2** | 142 ± 3.8 | 138 ± 2.3 | 128 ± 2.1** | 131 ± 2.6** |
| 5RM | 179 ± 4.0 | 173 ± 2.3 | 163 ± 2.0** | 160 ± 3.4** | 141 ± 2.2 | 137 ± 2.8 | 133 ± 1.5 | 137 ± 3.3 |

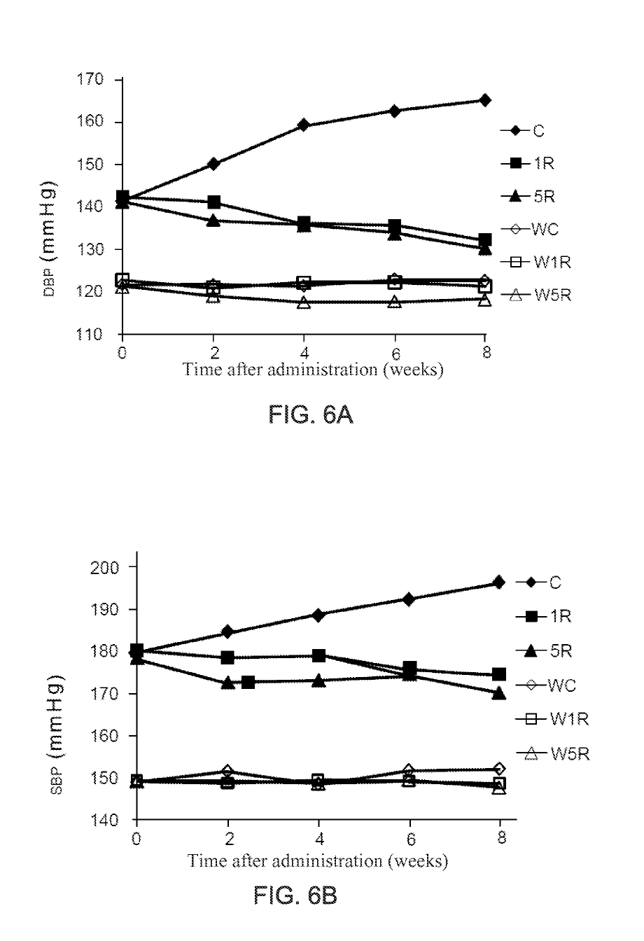

Please refer to FIG. 6A and FIG. 6B, there are respectively shown statistical data plots for systolic blood pressure (SBP) and diastolic blood pressure (DBP) of the SHRs and WKYs in the experiment groups listed in Table 6 and been treat with the chronic administration experiment. From FIG. 6A and FIG. 6B, it can find that the SBP value and the DBP value of the SHRs in C group gradually go up during 8-week chronic administration experiment; on the contrary, the SBP value and the DBP value of the SHRs in 1R group and 5R group gradually decrease during 8-week chronic administration experiment. Moreover, from FIG. 6A and FIG. 6B, it can also find that the DBP value of the WKYs in W1R group and W5R group show no obvious discrepancy comparing to the SBP value and the DBP value of the WKYs in WC group. Therefore, the experiment data of FIG. 6A and FIG. 6B have proven that: (1) the chronic administration of RMD can indeed lower the BP (blood pressure) value of a spontaneous hypertensive rat; and (2) the chronic administration of RMD would not affect the BP (blood pressure) value of a normotensive rat. The BP data for SHRs and WKYs in the experiment groups are integrated in following Table 8.

TABLE 8

| | SBP (mmHg) | | | | |
|---|---|---|---|---|---|
| Group | 0 week | 2 week | 4 week | 6 week | 8 week |
| WC | 149 ± 4.0 | 153 ± 1.7 | 150 ± 3.3 | 151 ± 2.5 | 151 ± 2.5 |
| W1R | 149 ± 2.7 | 149 ± 3.1 | 148 ± 3.7 | 148 ± 2.6 | 148 ± 2.6 |
| W5R | 149 ± 2.9 | 150 ± 2.4 | 149 ± 4.8 | 150 ± 3.1 | 150 ± 3.1 |
| C | 180 ± 7.5 | 180 ± 4.6 | 180 ± 8.2 | 181 ± 3.2 | 181 ± 3.2 |
| 1R | 180 ± 4.3 | 178 ± 3.6 | 168 ± 2.6* | 173 ± 4.7* | 173 ± 4.7* |
| 5R | 178 ± 4.0 | 173 ± 2.0* | 163 ± 3.2* | 170 ± 2.4* | 170 ± 2.4* |
| M | 180 ± 3.6 | 178 ± 5.4 | 171 ± 3.1 | 175 ± 7.6 | 175 ± 7.6 |
| 1RM | 178 ± 1.9 | 179 ± 4.3 | 164 ± 3.8** | 164 ± 3.2** | 164 ± 3.2** |
| 5RM | 179 ± 4.0 | 173 ± 2.3 | 163 ± 2.0** | 160 ± 3.4** | 160 ± 3.4** |
| | DBP (mmHg) | | | | |
| Group | 0 week | 2 week | 4 week | 6 week | 8 week |
| WC | 122 ± 3.6 | 122 ± 4.2 | 121 ± 3.6 | 123 ± 2.1 | 123 ± 1.9 |
| W1R | 123 ± 2.8 | 121 ± 3.0 | 122 ± 3.4 | 122 ± 1.6 | 121 ± 3.2 |
| W5R | 121 ± 2.9 | 119 ± 3.2 | 118 ± 3.7 | 118 ± 3.2 | 118 ± 2.3 |
| C | 141 ± 4.0 | 150 ± 4.0 | 159 ± 3.9 | 163 ± 2.8 | 165 ± 3.2 |
| 1R | 142 ± 3.3 | 141 ± 2.4 | 136 ± 3.4 | 136 ± 4.8* | 132 ± 4.4* |
| 5R | 141 ± 2.2 | 137 ± 2.9 | 136 ± 3.0 | 134 ± 1.9* | 130 ± 2.8* |
| M | 143 ± 2.9 | 145 ± 3.6 | 140 ± 3.8 | 139 ± 1.9 | 135 ± 2.7 |
| 1RM | 142 ± 3.8 | 143 ± 3.2 | 136 ± 3.2 | 132 ± 3.3** | 130 ± 2.0** |
| 5RM | 141 ± 2.2 | 136 ± 3.6** | 134 ± 2.3** | 130 ± 3.8** | 128 ± 4.5** |

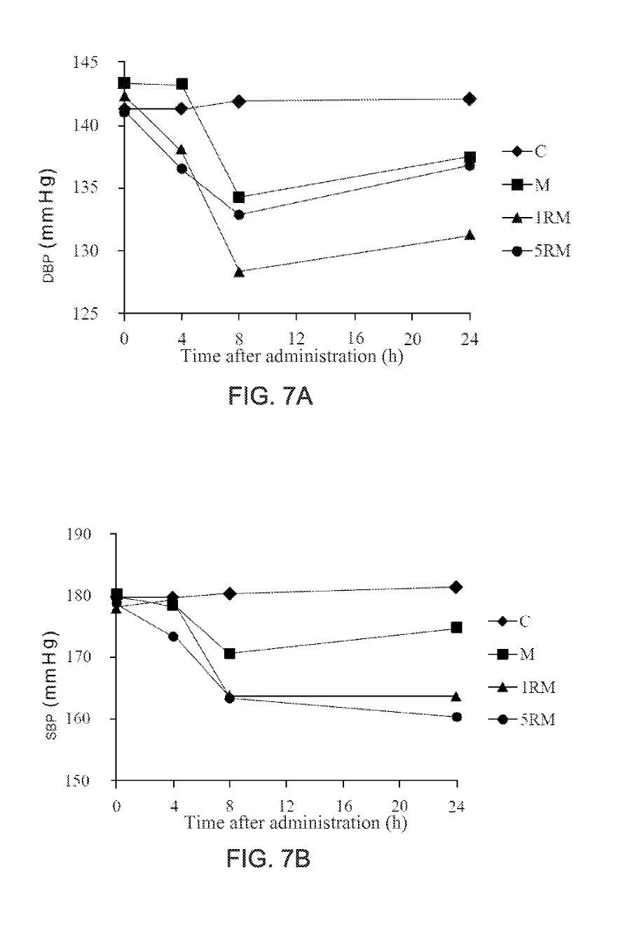

Furthermore, please refer to FIG. 7A and FIG. 7B, there are respectively shown statistical data plots for systolic blood pressure (SBP) and diastolic blood pressure (DBP) of the SHRs in the C group, M group, 1RM group, and 5RM group been treat with the one single oral administration. The SBP value and the DBP value of the SHRs in M group, 1RM group and 5RM group, as shown in FIGS. 7A and 7B, obviously decrease at 8 hr and 24 hr comparing to the SBP value and the DBP value of the SHRs in C group. Wherein the DBP decreasing difference of the SHRs in 5RM group is greater than the DBP decreasing difference of the SHRs in M group; moreover, the SBP decreasing differences of the SHRs in 1RM group and 5RM group are greater than the SBP decreasing difference of the SHRs in M group. Therefore, the experiment data of FIG. 7A and FIG. 7B have proven that: (1) the single one oral administration of the combination of 1×RMD and amlodipine (0.4 mgkg⁻¹day⁻¹) can indeed lower the BP (blood pressure) value of a spontaneous hypertensive rat; and (2) the single one oral administration of the combination of 5×RMD and amlodipine (0.4 mgkg⁻¹day⁻¹) can indeed lower the BP (blood pressure) value of a spontaneous hypertensive rat.

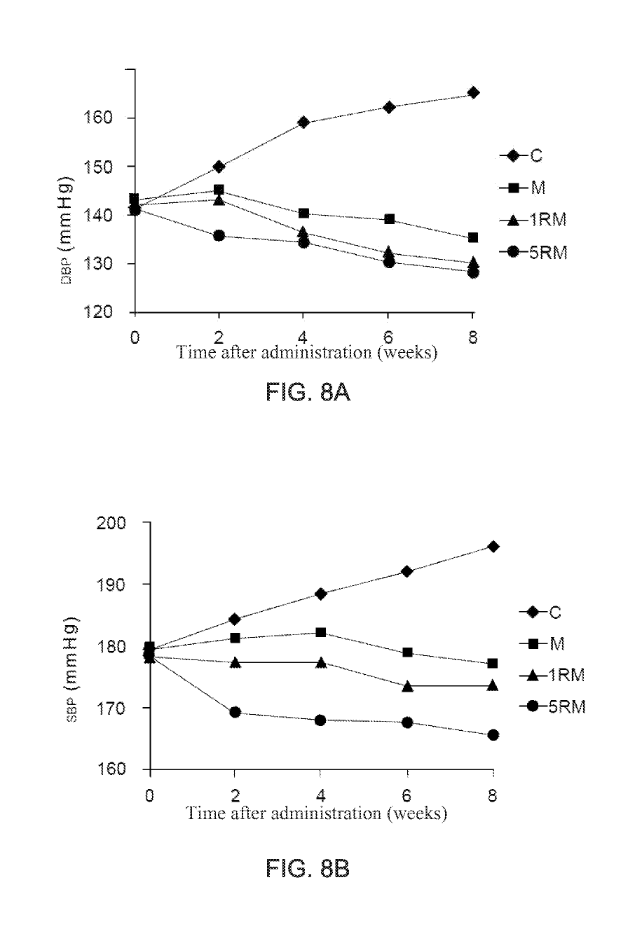

Eventually, please refer to FIG. 8A and FIG. 8B, there are respectively shown statistical data plots for systolic blood pressure (SBP) and diastolic blood pressure (DBP) of the SHRs in the C group, M group, 1RM group, and 5RM group been treat with the chronic administration experiment. From FIG. 8A and FIG. 8B, it can find that the SBP value and the DBP value of the SHRs in C group gradually go up during 8-week chronic administration experiment; on the contrary, the SBP value and the DBP value of the SHRs in M group, 1RM group and 5RM group gradually decrease during 8-week chronic administration experiment. Wherein the DBP decreasing differences of the SHRs in 1RM and 5RM groups are greater than the DBP decreasing difference of the SHRs in M group starting from 6-th week; moreover, the SBP decreasing differences of the SHRs in 1RM and 5RM groups are greater than the SBP decreasing difference of the SHRs in M group starting from 6-th week. Therefore, the experiment data of FIG. 8A and FIG. 8B have proven that: (1) the chronic administration of the combination of 1×RMD and amlodipine (0.4 mgkg⁻¹day⁻¹) can indeed lower the BP (blood pressure) value of a spontaneous hypertensive rat; and (2) the chronic administration of the combination of 5×RMD and amlodipine (0.4 mgkg⁻¹day⁻¹) can indeed lower the BP (blood pressure) value of a spontaneous hypertensive rat.

Continuously, please refer to following Table 9, which record blood lipids data of SHRs and WKYs in the 9 groups listed in the Table 6. From Table 9, it can find that, comparing to the concentrations of Triglycerides, Cholesterol, Low-density lipoprotein cholesterol (HDL-C), Cholesterol/HDL-C ratio in the blood of SHRs and WKYs in C group, WC group and M group, the concentrations of Triglycerides, Cholesterol, Low-density lipoprotein cholesterol (HDL-C), Cholesterol/HDL-C ratio in the blood of SHRs and WKYs in W1R group, W5R group, 1R group, 5R group, 1RM group, and 5RM group are obviously decreased. Therefore, the experiment data of Table 9 have proven that: (1) the administration of the combination of 1×RMD and amlodipine (0.4 mgkg⁻¹day⁻¹) can indeed lower the concentration of Triglycerides, Cholesterol, Low-density lipoprotein cholesterol (HDL-C), Cholesterol/HDL-C ratio of SHRs and WKYs rat; and (2) the administration of the combination of 5×RMD and amlodipine (0.4 mgkg⁻¹day⁻¹) can indeed lower the concentration of Triglycerides, Cholesterol, Low-density lipoprotein cholesterol (HDL-C), Cholesterol/HDL-C ratio of SHRs and WKYs rat.

TABLE 9

| | Triacylglycerol | Cholesterol | HDL-C | LDL-C | |
|---|---|---|---|---|---|
| Groups | (mg/dL) | | | | Cholesterol/HDL-C |
| WC | 74.4 ± 7.2 | 79.3 ± 4.2 | 55.7 ± 2.4 | 10.8 ± 1.1 | 1.4 ± 0.1 |
| W1R | 64.5 ± 6.2* | 74.2 ± 2.1 | 60.1 ± 3.1* | 8.5 ± 1.2* | 1.2 ± 0.0 |
| W5R | 62.7 ± 6.3* | 71.8 ± 2.5* | 59.1 ± 3.6* | 7.5 ± 2.0* | 1.2 ± 0.1 |
| C | 74.8 ± 4.1 | 84.1 ± 5.3 | 57.2 ± 4.5 | 11.5 ± 0.7 | 1.5 ± 0.1 |
| 1R | 67.5 ± 4.4** | 74.5 ± 4.1** | 62.8 ± 3.9** | 9.6 ± 0.7** | 1.2 ± 0.1** |
| 5R | 63.5 ± 4.7** | 73.5 ± 4.4** | 64.9 ± 3.4** | 8.2 ± 0.4** | 1.1 ± 0.1** |
| M | 69.3 ± 6.8 | 79.8 ± 3.6 | 60.3 ± 1.9 | 10.0 ± 0.7** | 1.3 ± 0.1 |
| 1RM | 64.5 ± 4.7** | 72.8 ± 4.5** | 62.6 ± 3.6** | 8.5 ± 1.2** | 1.1 ± 0.0** |
| 5RM | 63.1 ± 5.6** | 69.2 ± 3.2** | 61.1 ± 4.6** | 7.8 ± 0.8** | 1.1 ± 0.0** |

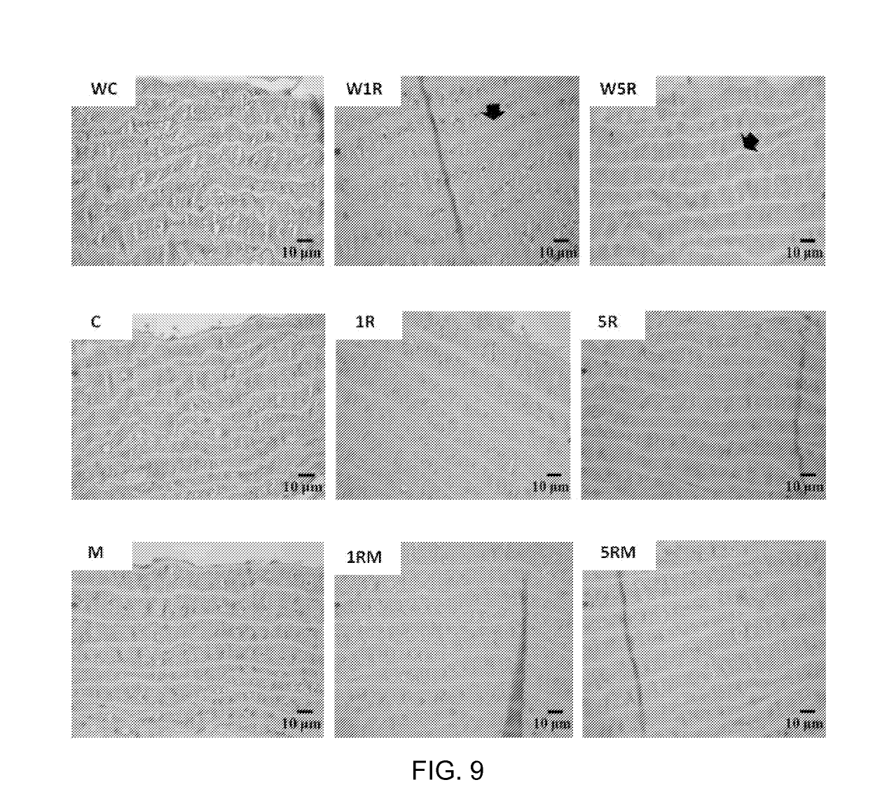

With reference to FIG. 9, which illustrate the histologic section images of the artery of the SHRs and the WKYs in the experiment group of Table 6. From FIG. 9, it can find that the vessel wall fibrins of the SHRs in 1R group and 5R group reveal smooth and order arrangement comparing with the vessel wall fibrin of the SHRs in C group. Moreover, the vessel wall fibrins of the WKYs in W1R group and W5R group reveal smooth and order arrangement comparing with the vessel wall fibrin of the WKYs in WC group. Thus, the histologic section images of the artery have proven that the RMD indeed includes the functionality to prevent Hypertension.

Continuously, please refer to following Table 10, which record the heart rate data of SHRs and WKYs in the experiment groups listed in Table 6. From Table 10, it can find that the heart rate of SHRs in 1R, 5R, 1RM, and 5RM groups are normal comparing to the heart rate of SHRs in C group. Moreover, the heart rate of SHRs in W1R and W5R groups are normal comparing to the heart rate of WKYs in WC group. Therefore, the experiment data of Table 10 have proven that: (1) the administration of the combination of 1×RMD (or 5×RMD) would not cause any adverse effects to the heart rate of SHRs and WKYs; and (2) the administration of the combination of 1×RMD (or 5×RMD) and amlodipine (0.4 mgkg⁻¹day⁻¹) would not cause any adverse effects to the heart rate of SHRs and WKYs.

TABLE 10

| | Heart rate (bpm) | | | | | | | |
|---|---|---|---|---|---|---|---|---|
| Groups | 0 hr | 4 hr | 8 hr | 24 hr | 2 week | 4 week | 6 week | 8 week |
| WC | 382 ± 5.0 | 386 ± 6.3 | 382 ± 4.2 | 382 ± 5.0 | 385 ± 4.8 | 383 ± 7.0 | 386 ± 4.7 | 386 ± 4.7 |
| W1R | 382 ± 4.9 | 381 ± 6.8 | 379 ± 4.8 | 382 ± 5.5 | 386 ± 5.0 | 388 ± 4.9 | 384 ± 5.0 | 383 ± 3.7 |
| W5R | 383 ± 5.2 | 381 ± 5.6 | 380 ± 4.3 | 382 ± 5.9 | 382 ± 6.3 | 386 ± 6.9 | 381 ± 3.9 | 381 ± 5.6 |
| C | 403 ± 4.8 | 402 ± 4.2 | 403 ± 5.2 | 401 ± 5.3 | 403 ± 4.8 | 404 ± 3.8 | 404 ± 4.5 | 403 ± 4.8 |
| 1R | 401 ± 4.2 | 402 ± 3.3 | 407 ± 2.4 | 401 ± 4.3 | 401 ± 4.3 | 402 ± 3.3 | 407 ± 2.4 | 401 ± 4.3 |
| 5R | 408 ± 7.7 | 402 ± 3.4 | 403 ± 5.5 | 402 ± 6.6 | 402 ± 3.1 | 404 ± 4.8 | 403 ± 5.6 | 401 ± 4.9 |
| M | 402 ± 6.9 | 401 ± 6.5 | 400 ± 4.8 | 402 ± 4.9 | 402 ± 6.7 | 404 ± 4.8 | 403 ± 6.1 | 402 ± 3.0 |
| 1RM | 405 ± 20.9 | 401 ± 4.2 | 401 ± 4.9 | 403 ± 5.5 | 402 ± 3.3 | 407 ± 2.4 | 401 ± 4.3 | 393 ± 16.3 |
| 5RM | 408 ± 7.7 | 402 ± 4.3 | 402 ± 6.5 | 401 ± 6.2 | 403 ± 3.5 | 403 ± 4.4 | 403 ± 5.6 | 403 ± 3.5 |

With reference to following Table 11, which record the liver function indexes data of SHRs and WKYs in the experiment groups listed in Table 6. From Table 11, it can find that the liver function indexes of SHRs in 1R, 5R, 1RM, and 5RM groups are normal comparing to the liver function indexes of SHRs in C group. Moreover, the liver function indexes of SHRs in W1R and W5R groups are normal comparing to the liver function indexes of WKYs in WC group. Wherein the liver function indexes data include: aspartate aminotransferase (AST), alanine aminotransferase (ALT), alkaline phosphatase, albumin, globulin, γ-Glutamyltransferase (γ-GT), total protein, and A/G ratio.

TABLE 11

| | | | Alkaline | | | | Total | |
|---|---|---|---|---|---|---|---|---|
| | AST | ALT | phosphatase | Albumin | Globulin | γ-GT | protein | A/G |
| Groups | (U/L) | | (IU/L) | (g/dL) | | | | ratio |
| WC | 124.5 ± 4.8 | 87.3 ± 8.2 | 130.3 ± 7.6 | 5.1 ± 0.2 | 2.4 ± 0.1 | 0.3 ± 0.1 | 7.6 ± 0.2 | 2.1 ± 0.2 |
| W1R | 122.5 ± 5.3 | 79.2 ± 4.5 | 131.3 ± 5.6 | 5.1 ± 0.1 | 2.5 ± 0.1 | 0.2 ± 0.1 | 7.6 ± 0.3 | 2.0 ± 0.3 |
| W5R | 117.2 ± 7.4 | 81.7 ± 4.4 | 133.8 ± 2.7 | 5.1 ± 0.1 | 2.5 ± 0.1 | 0.2 ± 0.1 | 7.4 ± 0.3 | 2.0 ± 0.2 |
| C | 137.7 ± 7.9 | 82.3 ± 4.7 | 135.5 ± 4.6 | 5.2 ± 0.3 | 2.6 ± 0.1 | 0.3 ± 0.1 | 8.0 ± 0.4 | 2.0 ± 0.1 |
| 1R | 125.1 ± 9.8 | 89.1 ± 6.7 | 133.5 ± 3.1 | 5.4 ± 0.2 | 2.4 ± 0.2 | 0.2 ± 0.1 | 7.7 ± 0.2 | 2.3 ± 0.3 |
| 5R | 123.2 ± 9.1 | 78.1 ± 7.5 | 135.8 ± 4.1 | 5.5 ± 0.1 | 2.4 ± 0.1 | 0.2 ± 0.1 | 7.6 ± 0.2 | 2.3 ± 0.2 |
| M | 125.8 ± 4.6 | 85.7 ± 6.1 | 143.3 ± 7.6 | 5.4 ± 0.2 | 2.6 ± 0.1 | 0.2 ± 0.1 | 8.2 ± 0.1 | 2.1 ± 0.4 |
| 1RM | 131.2 ± 9.2 | 84.6 ± 7.8 | 138.1 ± 8.4 | 5.4 ± 0.3 | 2.4 ± 0.1 | 0.2 ± 0.1 | 7.8 ± 0.3 | 2.3 ± 0.3 |
| 5RM | 127.8 ± 7.4 | 81.7 ± 7.9 | 135.6 ± 7.6 | 5.5 ± 0.2 | 2.4 ± 0.2 | 0.2 ± 0.1 | 7.5 ± 0.2 | 2.3 ± 0.2 |

Continuously, please refer to following Table 12, which record the data of kidney function indexes, electrolytes indexes and creatine phosphokinase indexes of SHRs and WKYs in the experiment groups listed in Table 6. From Table 12, it can find that the kidney function indexes, the electrolytes indexes and the creatine phosphokinase indexes of SHRs in 1R, 5R, 1RM, and 5RM groups are normal comparing to the kidney function indexes, the electrolytes indexes and the creatine phosphokinase indexes of SHRs in C group. Moreover, the kidney function indexes, the electrolytes indexes and the creatine phosphokinase indexes of SHRs in W1R and W5R groups are normal comparing to the kidney function indexes, the electrolytes indexes and the creatine phosphokinase indexes of WKYs in WC group. Wherein the kidney function indexes include blood urea nitrogen (BUN), creatinine and uric acid, and the electrolytes indexes include sodium and potassium. Such creatinine and uric acid data prove that RMD administration would not damage rat's kidney. It is well know that, when the kidney is at a kidney failure situation, the concentration of creatinine and uric acid in blood serum would increase. Moreover, such CPK data prove that RMD administration would not cause any damages or injuries to SHRs' muscle. It is well know that, when the muscle is subject to damage, the concentration of CPK in blood serum would increase.

TABLE 12

| | | | | | | Creatinine |
|---|---|---|---|---|---|---|
| | BUN | Creatinine | Uric acid | Sodium | Potassium | phosphokinase |
| Group | (mg/dL) | | | (meq/L) | | (U/L) |
| WC | 20.5 ± 1.0 | 0.46 ± 0.1 | 4.3 ± 0.6 | 152.6 ± 0.2 | 7.9 ± 0.5 | 267.5 ± 12.4 |
| W1R | 21.7 ± 0.9 | 0.48 ± 0.1 | 4.9 ± 1.3 | 152.4 ± 0.8 | 8.1 ± 1.6 | 276.6 ± 21.3 |
| W5R | 23.6 ± 1.5 | 0.47 ± 0.1 | 4.5 ± 0.7 | 152.3 ± 0.7 | 7.6 ± 0.5 | 291.3 ± 24.5 |
| C | 23.9 ± 0.9 | 0.44 ± 0.1 | 5.8 ± 1.2 | 149.1 ± 1.1 | 8.2 ± 0.5 | 289.2 ± 10.0 |
| 1R | 23.5 ± 1.0 | 0.46 ± 0.1 | 6.1 ± 1.5 | 149.9 ± 1.2 | 8.1 ± 0.4 | 287.2 ± 11.5 |
| 5R | 21.9 ± 0.7 | 0.43 ± 0.1 | 5.8 ± 0.8 | 151.1 ± 1.4 | 7.8 ± 0.6 | 279.7 ± 23.3 |
| M | 23.7 ± 0.8 | 0.48 ± 0.1 | 5.8 ± 0.6 | 152.4 ± 0.9 | 8.8 ± 0.4 | 287.0 ± 12.2 |
| 1RM | 23.1 ± 1.8 | 0.45 ± 0.1 | 5.7 ± 0.5 | 150.5 ± 0.7 | 8.6 ± 0.6 | 290.5 ± 12.6 |
| 5RM | 23.5 ± 1.2 | 0.51 ± 0.0 | 6.4 ± 0.8 | 149.1 ± 1.1 | 8.5 ± 0.8 | 289.6 ± 12.7 |

Thus, through above descriptions, the functionality to lower hypertension of RMD has been proven by the experiment data presented above. Next, for determining the compositions of the RMD, the extracting experiment is also completed. To extract the compositions for the RMD, 2.2 g powdered RMD is extracted by using deionized water under 60° C. for 30 min. Therefore, a RMD extract is obtained through filtering process, and the composition of the RMD extract is determined by using HPLC method (high-performance liquid chromatography). According to HPLC result, it is able to know that the RMD include monascin of 6.82 mg/g and γ-aminobutyric acid (GABA) of 1.02 mg/g. Moreover, according to each of experiment results, it can further confirm that the blood pressure reducing composition of the present invention includes the monascin ranged from 3 mg/g to 6.82 mg/g.

Thus, through above descriptions, the RMD including 3 mg/g˜6.82 mg/g monascin has been proven to be a safe blood pressure reducing composition, without causing any damages or injuries to liver, kidney and muscle. Next, for the nucleotide sequence of the Monascus purpureus NTU 568 can be formed by treating the RAPD (Random Amplification of Polymorphic DNA) and the PCR (Polymerase Chain Reaction) process to a plurality of specific primers, the specific primers will be introduced in follows.

As the following table 13 shows, the primers designed by the software of Geneious 4.5.8 are recorded. According to the following Sequence Listing, the nucleotide sequence of primer PKSα F is defined as SEQ ID NO 4 and has 19 bp sequence length, the nucleotide sequence of primer PKSα R is defined as SEQ ID NO 5 and has 19 bp sequence length, the nucleotide sequence of primer PKSδ F is defined as SEQ ID NO 6 and has 20 bp sequence length, and the nucleotide sequence of primer PKSδ R is defined as SEQ ID NO 7 and has 20 bp sequence length. Moreover, according to the following Sequence Listing, the nucleotide sequence of primer PKSγ F is defined as SEQ ID NO 8 and has 20 bp sequence length, and the nucleotide sequence of primer PKSγ R is defined as SEQ ID NO 9 and has 20 bp sequence length.

TABLE 13

| Primer | Sequence | Tar- |
|---|---|---|
| ID | (5′→3′) | get |
| PKSα F | GACTGCGGTCATCCGGCCC | PKSα |
| PKSα R | GCGTGTCCCCGGAGCTACA | |
| PKSδ F | GCGAGCCAACCGTCTGGACC | PKSδ |
| PKSδ R | CGAGACGACCACCGTTGCCC | |
| PKSγ F | GCGAGCCAACCGTCTGGACC | PKSγ |
| PKSγ R | CGAGACGACCACCGTTGCCC | |

Continuously, the primers listed in the table 13 are executed RAPD through PCR process, wherein the polymerase chain reaction cocktail contains 3 ng DNA, 20 nM primers, a 1× Exsel reaction buffer, 0.5U Exsel DNA polymerase (Bertec Enterprise, Taipei, Taiwan), and 100 M dNTPs. The reaction conditions of the PCR is as described: (1) 35-cycle processes with 95° C. (5 min) for heating, 95° C. (30 sec) for heating and −62° C. (1 min) for cooling; and (2) 70° C. (10 min) for reaction. Moreover, after completing the PCR process, it is able to execute the electrophoresis analysis for the PCR products by using 1% agarose gel, wherein the MISSION BIOTECH Co. Ltd. is commissioned to complete the electrophoresis analysis. Therefore, the electrophoresis analysis and genome sequencing results are recorded in following table 14. From table 14, it is able to confirm and prove that the PKSα PKSγ and PKSδ are indeed the novel ovel gene fragment (sequence) for the Monascus purpureus NTU 568.

TABLE 14

| | Sequence | |
|---|---|---|
| PKS | Length | |
| ID | (bp) | Sequence |
| α | 1390 | GACTGCGGTCATCCGGCCCAGGAAACCAG |
| | | AATGGATATCTGGCGCCTTCTAGAACCTGG |
| | | ATAGTGGCCGGATCCCTCGCGCTGGGAGC |
| | | CTGGGTGACTATGAGGGAGGCGTTGGAA |
| | | CCGGAGGCGCCATAATTATTGATGAGGGC |
| | | CGCGCGGAAGTCCTCGTTCCAGGGCGTCA |
| | | GCTTGGTGGCAATCTTCATGTTATGTTCTG |
| | | GCAAGGCTTTTATGGATGGATTCATGGTGG |
| | | TAAAGCTTGCCTGGGGTGGGATGTAACCTT |
| | | CATTAATCATGAGGAGCACCTTGATGAGGG |
| | | AAATGACCCCTGACGTACACTCGGTATGTC |
| | | CGATGAGGCCCTTGACAGAGCCAAAGTGC |
| | | AGTGGTGTTGAGCGATTGGGGCCCCCAAGT |
| | | ACTCTCAGGATACTCTCATATTCTGCTGGGT |
| | | CTCCCACAGGAGTGCCAGTGCCGTGAGCTT |
| | | CAACGACAGTAATCTGTTTAGGCACCAGAT |
| | | GGGCCTCCCTGGTAACGTCCTTGAAGAGCT |
| | | CTGAAAGGGAGGGCGAGTTTGGCACGAAG |
| | | ATTGGGGTGCAGTTCTGGTTTTGATAGACA |
| | | GCGGTGCTCGCAATGGTCCCCAGGATCTGG |
| | | TCGCCGTCCTCAATTGCAGTGCTGAGCTTC |
| | | TTCAAGAAGACAGCAGCAATGCCTTCACC |
| | | GCGACAATAGCCATCTGCATGAGCGTCGA |
| | | ATGGCTTGCATTGGCCCGTTGGACTCAGGA |
| | | AGGACGCCCCTGCCAAGTTCTGGAACCAG |
| | | AGAGGATTCGTCATTACATTCGTACCACCG |
| | | GCCAGGGCAGCGGTACACTCGCCGCTGAG |
| | | GATAGCTTTGCAGGCCTGATGAACTGCTA |
| | | CAGCGGACGAGGAGCATGCAGTGTCGATG |
| | | GTCAGGCCAGGACCGGTCCAGCCGAAGTA |
| | | GTGGCTGATCTTTCCTGCAATGAAGCTCTT |
| | | CAGGTTGCCAGTGGCCGAGAAGGCATTCG |
| | | GAGCATGGCAGGCAATGTTGTTCTCATAGT |
| | | CCGCAGCGCAAACGCCAATATAGCACCCA |
| | | ATCTGCTTGTCAACGCTGGGGTTGCAGAAA |
| | | TATCCCGACTGTTCGACAGCCTGATAGGCGA |
| | | TTTGCAGCATGTGGCGCTGCTGAGGATCCG |
| | | TCGAGGCAATCTCTCGCGGGCTCTTCTTGA |
| | | AGAACTTGTGATCAAAGGCATCGTGGTCTC |
| | | GGATAAAGTTTCCAAACCACTTCCGTTTCG |
| | | TATCGAGCTCGCGGAATATTGTGTCGAAGG |
| | | TAAAGCGTTCCTTGGGTACTTCCTGGTGC |
| | | TGTGACTCCCCCCTGCAGAGCAAGTCCCA |
| | | GAACCCTTCGAGGTCATCTGCACCGGCCA |
| | | CCTTACACGACATGCCAATGACGGCGATG |
| | | TCGTTTTCGTCGACCGCATGGGCGTATTT |
| | | CAAAGCAGATGTAGCTCCGGGGACACGCA |
| δ | 1024 | GCGAGCCAACCGTCTGGACCAACTCGACC |
| | | GTCATTCTCTCAAAGTCCTGACGGATCTGC |
| | | CCTCCTATCCCTGGATGCATTCCCTCCGGTT |
| | | CTGGTACGAGTCTCGTCTAAGCTATGACTAT |
| | | CGCCATCGATCACACCCTCGTCACCACCTG |
| | | GTAGGGGCTCCCACGGCGGATCACAACGCA |
| | | CTGGAGCCGAGATGGAGAAACTACCTGCGG |
| | | GTCTCCGAGAGCCCCTGGATACGCGAGCAC |
| | | GTCGTTCAGTCTCGCATAATCTACCCAGGTG |
| | | CGGGATTCATCGTGATGGCAATCGAGGCTG |
| | | CCGCTCAGCTGGCGGATTCGTCGAAGAAGG |
| | | TCAAGGGGTTCGAGCTGCGAGATGTCCAGA |
| | | TCAACCGGGCATTGCAGGTGCCGGAAGGCG |
| | | AAGAAGGCGTTGAAACCATACTCCACCTGC |
| | | GTCCGTATCAGGCGCAGGGCCTCACCAAGG |
| | | GCTCGCACTGGGACGAGTTCGTCATCTATT |
| | | CCTACCAGTCAACGCAGGGCTGGCAAGAC |
| | | CACGCGCGTGGCTTGATCGTGACACACTA |
| | | CCACAGCAACAAGGCGGGGTTTGATCTGC |
| | | ATCGGGAAGACGAGATACAGCTGCAGATG |
| | | CATCGGGAGCAATACCTGAGATCCTCTGGG |
| | | CTATGCTTGTCGACAATCGAACTGGATGCG |
| | | TTCTACGATCGCCTCGGCCAGATGGGCATG |
| | | GAATTTGGTCCGGCATTCCGCAACCTGTCG |
| | | AGCATCCGACACTGCAACGGCCAGAGTGT |
| | | CTGTCAGCTGCGTATTCCAGACACCAAAG |
| | | TGCAGATGCCAGACGAGTTTGAGTTTAAG |
| | | CATGTTATTCACCCCATCACGCTGGATAAC |
| | | ATCTTCCACATGGTTCTGCCCTCTCGAGTA |
| | | GGATCGGGTGCATCGATGAGGGATGCGCA |
| | | TGTTCCGGTCTCCCTGCAGAGTCTGTATA |
| | | TTGCTGCCGATATAAAAAGCAACCCTGGG |
| | | ACCCTCCTTACAGGCCAATCCACCATTAC |
| | | GCATGAGGACGACAGCGGTTTTGGGGCA |
| | | ACGGTGGTCGTCTCG |
| γ | 1096 | AGCACCTCGGAGCAACGGTTCTTGCGATTG |
| | | CAAATACAATGAGTGGGAAACTGAGCTTGC |
| | | TCAATTCCTTCCCGGATTCAACTGTTCTCAC |
| | | CCTGGATGAAATTACGAATTCGAGCACTCA |
| | | GACGTTCGGACGAGCGGACGTCATCCTGAG |
| | | CAACCATGGGGTCAACCCAAGATGGTATCA |
| | | TGGGGAATTATTAGGGCCATGCGGGCGCTT |
| | | TATCGATTACTCTGACATTGAAGGTACCAC |
| | | GAGTCATATTGCAGATGACAGTCAGGCTGA |
| | | TGAAATCTTGATCCATAGCGAAGTCTGTGC |
| | | CAGGATTGACCTCGACTGTCTTCTCAAGCA |
| | | TCGACCAGTGCTGGTTTCTGAAGTCTTAGA |
| | | AGTCGCGCACAATTTGGTTAGAGAGAGAA |
| | | TCGTGAATATTGGAGGCAAAGAGCCCAAG |
| | | ATATTCTCATTCTCACAACTACAACTTGCA |
| | | TTTGACCACCTGGCATCTATGCAGGACACT |
| | | GTGCCTACTATCATCACGGCCGAAGACGGC |
| | | TGTCAAGTCAGCGTCTCGCCACCATCCTTC |
| | | GGCTCCACCCCATTCATCTTCTCCCCGGAC |
| | | AAAGTGTATCTTCTCGTGGGGGGCCTGAGC |
| | | GGTCTTGGCCTTGAGCTGGCCGAATGGATG |
| | | GTGCTCCGTGGCGCGCGTCAGCTTGCTTTC |
| | | ATGTCTCGATCGGGTGCAGGAAACGCCGCT |
| | | GCGACTGCTATGCTGGCGAGATTGGCGGCA |
| | | AAAGGGGCGCGAACAACGGTGTACCGATG |
| | | CGATGTGACCGATTTCTCCGCAGTGGGACA |
| | | ATGCATCATGCAGATAGGGCCTCAGTTAGG |
| | | CGGTATTTTCCATGCCGCTGCGGTGATTGA |
| | | TGACTGCCCCCTGCAGCAGATGTCCGTTTC |
| | | CCAATGGTGTCGCACAATCTCGCCCAAGGT |
| | | CCGCGGAGCAGACAACCTTGATCGAGCAA |
| | | CAGCAGGCATGGACTTGGACTTTTTCATCT |
| | | GCTTCTCCTCTGCCTCAGCAGTGGTTGGAA |
| | | CCAAGGCCCAGGCAAGCTATGTGGCCGGC |
| | | AACACCTACATGGACGCCCTGATGCGGAG |
| | | CCGTCGACAGCGCGGACTAAGTGGCACGG |
| | | CCATTAATATCGGCATGGTGATAGGGATTG |
| | | GTCTGGTCGCTGCGGATGCTAAGCTTGAG |
| | | GCAAGCATGAAACGGACTGGTTTCGATCC |
| | | GGTCAATGAGTATGAATTCTTCTGTCTGAT |
| | | AGAAGAGGCAGTTCAGACAGGACGCTCGC |
| | | TGACGACCTCCGACGACGGGAACATGGAG |
| | | AGTTTCCGGATTGTTACTGGGGCTCGCGTG |
| | | ACAGGGCCACAGTGCT |

Thus, through the descriptions, the mutant of Monascus purpureus NTU 568, nucleotide sequence for Monascus purpureus NTU 568 and primers for nucleotide sequence of Monascus purpureus NTU 568 of the present invention has been completely introduced and disclosed; in summary, the present invention has the following advantages: In the present invention, the nucleotide sequence for Monascus purpureus NTU 568 and the primers for the nucleotide sequence are proposed in order to facilitate the person skilled in Monascus purpureus filed capable of carrying out the strain (mutant) identification of the Monascus purpureus NTU 568 according to the present invention. Moreover, the person skilled in Monascus purpureus filed can also rapidly complete the strain (mutant) identification of the Monascus purpureus NTU 568 by using DNA molecular marker technology, without culturing any isolated Monascus purpureus strain or live Monascus purpureus bacteria.

Moreover, the present invention further includes the following advantages: (1) According to above-presented experiment data, the powdered red mold dioscorea (RMD) has been proven to be a composition having the functionality to prevent the vessel wall from illness and lower the blood pressure, such that the RMD can be applied to be a clinical treatment agent or a health food. (2) Moreover, above-presented experiment data also prove that RMD intake would not cause any burdens to humane body and induce any side effects; besides, RMD intake also would not cause any adverse effects for body weight, liver function, kidney function, muscle, and electrolyte balance.

The above description is made on embodiments of the present invention. However, the embodiments are not intended to limit scope of the present invention, and all equivalent implementations or alterations within the spirit of the present invention still fall within the scope of the present invention.

What is claimed is:

1. A blood pressure reducing composition, being a red mold dioscorea (RMD) manufacturing by way of inoculating a Monascus purpureus NTU 568 to a dioscorea substrate and then treating the inoculated dioscorea substrate with culturing and drying processes; wherein, an specific intake dosage of the blood pressure reducing composition for an adult user used to reduce the systolic blood pressure (SBP) and diastolic blood pressure (DBP) in a short period of 8 hr is ranged from 2.2 g to 11 g.

2. The blood pressure reducing composition of claim 1, wherein a specific weight percent of the blood pressure reducing composition in a daily diet amount of the adult user used for chronically reducing the systolic blood pressure (SBP) and diastolic blood pressure (DBP) is ranged between 0.2 wt % and 0.25 wt %.

3. The blood pressure reducing composition of claim 1, wherein the red mold dioscorea comprises a yellow pigment formed during the culturing process of the inoculated dioscorea substrate, and the yellow pigment is monascin ranged between 3 mg/g and 6.82 mg/g.

4. The blood pressure reducing composition of claim 1, wherein the Monascus purpureus NTU 568 has a nucleotide sequence of SEQ ID NO 1, SEQ ID NO 2 or SEQ ID NO 3, and the Monascus purpureus NTU 568 being deposited with Deutsche Sammlung von Mikroorganismen and Zellkulturen GmbH (DSMZ, Inhoffenstr. 7B, D-38124 Braunschweig, Germany) on Nov. 18, 2013, with the accession number of DSM 28072.

5. The blood pressure reducing composition of claim 4, wherein the nucleotide sequence of the Monascus purpureus NTU 568 can be formed by treating the RAPD (Random Amplification of Polymorphic DNA) and the PCR (Polymerase Chain Reaction) process to a plurality of specific primers.

6. The blood pressure reducing composition of claim 5, wherein the specific primers comprise a first nucleotide sequence of SEQ ID NO 4 or SEQ ID NO 5.

7. The blood pressure reducing composition of claim 6, wherein the specific primers further comprise a second nucleotide sequence of SEQ ID NO 6 or SEQ ID NO 7.

8. The blood pressure reducing composition of claim 7, wherein the specific primers further comprise a third nucleotide sequence of SEQ ID NO 8 or SEQ ID NO 9.

9. A primer for identifying the said Monascus purpureus NTU 568 of claim 1, being selected from the group consisting of:
(1) primer PKSα F:
(SEQ ID NO 4)
GACTGCGGTCATCCGGCCC;
(2) primer PKSα R:
(SEQ ID NO 5)
GCGTGTCCCCGGAGCTACA;
(3) primer PKSδ F:
(SEQ ID NO 6)
GCGAGCCAACCGTCTGGACC;
(4) primer PKSδ R:
(SEQ ID NO 7)
GCGTGTCCCCGGAGCTACA;
(5) primer PKSγ F:
(SEQ ID NO 8)
GCGAGCCAACCGTCTGGACC;
and
(6) primer PKSγ R:
(SEQ ID NO 9)
CGAGACGACCACCGTTGCCC.

10. The primer of claim 9, wherein the primer PKSα F or the primer PKSα R can be amplified to the nucleotide sequence of SEQ ID NO 1 after being processed the RAPD (Random Amplification of Polymorphic DNA) and the PCR (Polymerase Chain Reaction) process.

11. The primer of claim 9, wherein the primer PKSδ F or the primer PKSδ R can be amplified to the nucleotide sequence of SEQ ID NO 2 after being processed the RAPD (Random Amplification of Polymorphic DNA) and the PCR (Polymerase Chain Reaction) process.

12. The primer of claim 9, wherein the primer PKSγ F or the primer PKSγ R can be amplified to the nucleotide sequence of SEQ ID NO 3 by way of being processed the RAPD (Random Amplification of Polymorphic DNA) and the PCR (Polymerase Chain Reaction) process.


SEQUENCE LISTING (9 sequences)

SEQ ID NO: 1 - DNA, 1389 bp, Monascus purpureus
gactgcggtcatccggcccaggaaaccagaatggatatctggcgccttctagaacctggatagtggccggatccctcgcgctgggagcctgggtgactatgagggaggcgttggaaccggaggcgccataattattgatgagggccgcgcggaagtcctcgttccagggcgtcagcttggtggcaatcttcatgttatgttctggcaaggcttttatggatggattcatggtggtaaagcttgcctggggtgggatgtaaccttcattaatcatgaggagcaccttgatgagggaaatgacccctgacgtacactcggtatgtccgatgaggcccttgacagagccaaagtgcagtggtgttgagcgattggggcccccaagtactctcaggatactctcatattctgctgggtctcccacaggagtgccagtgccgtgagcttcaacgacagtaatctgtttaggcaccagatgggcctccctggtaacgtccttgaagagctctgaaagggagggcgagtttggcacgaagattggggtgcagttctggttttgatagacagcggtgctcgcaatggtccccaggatctggtcgccgtcctcaattgcagtgctgagcttcttcaagaagacagcagcaatgccttcaccgcgacaatagccatctgcatgagcgtcgaatggcttgcattggcccgttggactcaggaaggacgcccctgccaagttctggaaccagagaggattcgtcattacattcgtaccaccggccagggcagcggtacactcgccgctgaggatagctttgcaggcctgatgaactgctacagcggacgaggagcatgcagtgtcgatggtcaggccaggaccggtccagccgaagtagtggctgatctttcctgcaatgaagctcttcaggttgccagtggccgagaaggcattcggagcatggcaggcaatgttgttctcatagtccgcagcgcaaacgccaatatagcacccaatctgcttgtcaacgctggggttgcagaaatatcccgactgttcgacagcctgataggcgatttgcagcatgtggcgctgctgaggatccgtcgaggcaatctctcgcgggctcttcttgaagaacttgtgatcaaaggcatcgtggtctcggataaagtttccaaaccacttccgtttcgtatcgagctcgcggaatattgtgtcgaaggtaaagcgttccttgggtacttcctggtgctgtgactcccccctgcagagcaagtcccagaacccttcgaggtcatctgcaccggccaccttacacgacatgccaatgacggcgatgtcgttttcgtcgaccgcatgggcgtatttcaaagcagatgtagctccggggacacgca

SEQ ID NO: 2 - DNA, 1024 bp, Monascus purpureus
gcgagccaaccgtctggaccaactcgaccgtcattctctcaaagtcctgacggatctgccctcctatccctggatgcattccctccggttctggtacgagtctcgtctaagctatgactatcgccatcgatcacaccctcgtcaccacctggtaggggctcccacggcggatcacaacgcactggagccgagatggagaaactacctgcgggtctccgagagcccctggatacgcgagcacgtcgttcagtctcgcataatctacccaggtgcgggattcatcgtgatggcaatcgaggctgccgctcagctggcggattcgtcgaagaaggtcaaggggttcgagctgcgagatgtccagatcaaccgggcattgcaggtgccggaaggcgaagaaggcgttgaaaccatactccacctgcgtccgtatcaggcgcagggcctcaccaagggctcgcactgggacgagttcgtcatctattcctaccagtcaacgcagggctggcaagaccacgcgcgtggcttgatcgtgacacactaccacagcaacaaggcggggtttgatctgcatcgggaagacgagatacagctgcagatgcatcgggagcaatacctgagatcctctgggctatgcttgtcgacaatcgaactggatgcgttctacgatcgcctcggccagatgggcatggaatttggtccggcattccgcaacctgtcgagcatccgacactgcaacggccagagtgtctgtcagctgcgtattccagacaccaaagtgcagatgccagacgagtttgagtttaagcatgttattcaccccatcacgctggataacatcttccacatggttctgccctctcgagtaggatcgggtgcatcgatgagggatgcgcatgttccggtctccctgcagagtctgtatattgctgccgatataaaaagcaaccctgggaccctccttacaggccaatccaccattacgcatgaggacgacagcggttttggggcaacggtggtcgtctcg

SEQ ID NO: 3 - DNA, 1296 bp, Monascus purpureus
agcacctcggagcaacggttcttgcgattgcaaatacaatgagtgggaaactgagcttgctcaattccttcccggattcaactgttctcaccctggatgaaattacgaattcgagcactcagacgttcggacgagcggacgtcatcctgagcaaccatggggtcaacccaagatggtatcatggggaattattagggccatgcgggcgctttatcgattactctgacattgaaggtaccacgagtcatattgcagatgacagtcaggctgatgaaatcttgatccatagcgaagtctgtgccaggattgacctcgactgtcttctcaagcatcgaccagtgctggtttctgaagtcttagaagtcgcgcacaatttggttagagagagaatcgtgaatattggaggcaaagagcccaagatattctcattctcacaactacaacttgcatttgaccacctggcatctatgcaggacactgtgcctactatcatcacggccgaagacggctgtcaagtcagcgtctcgccaccatccttcggctccaccccattcatcttctccccggacaaagtgtatcttctcgtggggggcctgagcggtcttggccttgagctggccgaatggatggtgctccgtggcgcgcgtcagcttgctttcatgtctcgatcgggtgcaggaaacgccgctgcgactgctatgctggcgagattggcggcaaaaggggcgcgaacaacggtgtaccgatgcgatgtgaccgatttctccgcagtgggacaatgcatcatgcagatagggcctcagttaggcggtattttccatgccgctgcggtgattgatgactgccccctgcagcagatgtccgtttcccaatggtgtcgcacaatctcgcccaaggtccgcggagcagacaaccttgatcgagcaacagcaggcatggacttggactttttcatctgcttctcctctgcctcagcagtggttggaaccaaggcccaggcaagctatgtggccggcaacacctacatggacgccctgatgcggagccgtcgacagcgcggactaagtggcacggccattaatatcggcatggtgatagggattggtctggtcgctgcggatgctaagcttgaggcaagcatgaaacggactggtttcgatccggtcaatgagtatgaattcttctgtctgatagaagaggcagttcagacaggacgctcgctgacgacctccgacgacgggaacatggagagtttccggattgttactggggctcgcgtgacagggccacagtgct

SEQ ID NO: 4 - DNA, 19 bp, Artificial Sequence
  Synthetic oligonucleotides
gactgcggtcatccggccc

SEQ ID NO: 5 - DNA, 19 bp, Artificial Sequence
  Synthetic oligonucleotides
gcgtgtccccggagctaca

SEQ ID NO: 6 - DNA, 20 bp, Artificial Sequence
  Synthetic oligonucleotides
gcgagccaaccgtctggacc

SEQ ID NO: 7 - DNA, 19 bp, Artificial Sequence
  Synthetic oligonucleotides
gcgtgtccccggagctaca

SEQ ID NO: 8 - DNA, 20 bp, Artificial Sequence
  Synthetic oligonucleotides
gcgagccaaccgtctggacc

SEQ ID NO: 9 - DNA, 20 bp, Artificial Sequence
  Synthetic oligonucleotides
cgagacgaccaccgttgccc